(19) United States
(12) Patent Application Publication
(10) Pub. No.: US 2010/0217390 A1
(43) Pub. Date: Aug. 26, 2010

(54) ROTATOR CUFF BONE-TENDON ALLOGRAFT

(75) Inventor: James Lee Cook, Columbia, MO (US)

Correspondence Address: POLSINELLI SHUGHART PC, 700 W. 47TH STREET, SUITE 1000, KANSAS CITY, MO, 64112-1802, US

(73) Assignee: THE CURATORS OF THE UNIVERSITY OF MISSOURI, Columbia, MO (US)

(21) Appl. No.: 12/711,606

(22) Filed: Feb. 24, 2010

Related U.S. Application Data
(60) Provisional application No. 61/208,391, filed on Feb. 24, 2009.

(51) Int. Cl. A61F 2/08

(52) U.S. Cl. 623/13.14

(57) ABSTRACT

The invention provides a biomaterial and a method of selecting a biomaterial for a rotator cuff repair procedure. The biomaterial includes a bone-tendon allograft and the use of the biomaterial in rotator cuff repairs may provide immediate bone-tendon integrity and function, which may result in lower failure rates and enhanced clinical success.

### CROSS-REFERENCE TO RELATED APPLICATION

This application claims priority to U.S. Provisional Application Ser. No. 61/208,391 filed on Feb. 24, 2009, which is hereby incorporated by reference in its entirety.

### FIELD OF INVENTION

The present invention relates to a biomaterial that includes an intact bone-tendon unit for use in the repair of a rotator cuff. The present invention further relates to a method of selecting a biomaterial for the repair of a rotator cuff injury.

### BACKGROUND OF INVENTION

Rotator cuff injuries are common, especially among athletes. The tendons at the ends of the rotator cuff muscles may become torn, leading to pain and restricted movement of the arm. A torn rotator cuff may occur following an acute trauma to the shoulder or through chronic wear of the tendons. Injuries of the rotator cuff are commonly associated with activities that require repeated overhead motions or forceful pulling motions and are relatively common injuries among athletes performing repetitive throws. “Wear and tear” rotator cuff problems commonly occur in the elderly.

Current treatments of rotator cuff injuries include noninvasive treatments and surgical treatments. Noninvasive treatments, such as the conservative R.I.C.E. treatment (rest, ice, compression, and elevation), are recommended for minor or moderate rotator cuff tears; conservative care typically results in a reduction in the patient's symptoms. However, many patients, especially with full thickness rotator cuff tears, still suffer disability and pain despite non-surgical therapies. For massive tears of the rotator cuff, surgery has shown more functional and durable outcomes in recent years.

The type of orthopaedic surgery performed depends on the size, shape, and location of the tear of the rotator cuff. Three existing approaches are typically available for surgical repair: (1) arthroscopic repair, in which a fiber optic scope and small, pencil-sized instruments are inserted through small incisions, and the surgeon performs the repair under video control; (2) mini-open repair; and (3) open surgical repair, in which a traditional open surgical incision is performed.

Regardless of the approach, the current surgical repairs mainly entail the use of soft tissue scaffolds, such as tendon transfer. However, the current biomaterials used in soft tissue repair techniques are not “span” or “structural” grafts and as a result have significant failure rates (up to 90% in some studies). In addition, current tissue repair techniques do not re-establish normal bone-tendon or tendon-muscle junctions, and the quality of the recipient tissue used in these repairs is often poor.

Using a “span” or “structural” graft with a normal bone-tendon junction composed of good quality tissue of the same type as being repaired would potentially overcome the major drawbacks of current tissue repair treatments. This surgical approach has been used with very high rates of successful long-term functional outcomes in anterior cruciate ligament (ACL) surgery. In this surgical approach, the injured ACL is replaced with cadaveric bone-ligament-bone or bone-tendon-bone constructs to repair or replace any defective structure. However, the surgical approach is infrequently utilized for surgical rotator cuff repair.

A need exists to provide new and improved biomaterials with intact bone-tendon allografts for rotator cuff repair surgery. Using these biomaterials, an improved bone-tendon allograft technique for the repair of a rotator cuff injury may be developed. Due to a more optimal strength and function of the biomaterial at the bone-tendon and tendon-muscle junctions, such an allograft technique may result in lower failure rates and greater long-term clinical success.

### SUMMARY OF INVENTION

Embodiments of the invention provide a biomaterial for use in a rotator cuff repair, which includes an intact bone-tendon unit. The bone-tendon unit includes a bone block attached to a tendon, provided however that the intact bone-tendon unit consists of tissues other than knee tissues or ankle tissues. Because the biomaterial includes an intact bone-tendon unit, the biomaterial overcomes several limitations of existing rotator cuff repair materials such as tendon allografts that must be attached to the native bone tissue using fixation devices such as sutures or screws. For example, the strength of attachment of the tendon to the bone block of the biomaterial is comparable to that of a healthy tendon and is not affected by the rate of healing. The biomaterial may be attached to the native bone material using existing fasteners such as cannulated screws, which impart immediate strength to the attachment and accelerate the recovery process.

The invention also provides a method of selecting a biomaterial that includes a bone block attached to a tendon for use in a rotator cuff repair that includes selecting a biomaterial that includes a tendon that is a rotator cuff tendon. Because the biomaterial is essentially similar in morphological and biomechanical properties to the injured rotator cuff tissue, the biomaterial functions in an essentially similar manner to uninjured rotator cuff tissue. As a result, the overall function of a rotator cuff repaired using the biomaterial may be similar to a healthy uninjured rotator cuff.

### BRIEF DESCRIPTION OF DRAWINGS

FIG. 1 is a photograph of an exemplary bone-tendon allograft.

FIG. 2A is a photograph of a bone-tendon allograft.

FIG. 2B is a photograph of the tendon end of a bone-tendon allograft sutured to a native tendon.

FIG. 2C is a photograph of the bone block of a bone-tendon allograft attached to a native bone.

FIG. 2D is a radiograph image of a rotator cuff repaired using a bone tendon allograft obtained 12 weeks after surgery.

FIG. 3A is a photograph of the insertion of a tendon-only allograft.

FIG. 3B is a photograph of a tendon-only allograft sutured to a native tendon.

FIG. 3C is a radiograph image of a rotator cuff repaired using a tendon-only allograft obtained 12 weeks after surgery.

FIG. 4A is a photograph of the elevation of a native bone block in an IST autograft.

FIG. 4B is a photograph of the bone block of an IST autograft attached to a native bone.

FIG. 4C is a radiograph image of a rotator cuff repaired using an autograft obtained 12 weeks after surgery.

FIG. 5A is an ultrasound image of a bone-tendon allograft obtained 12 weeks after surgery.

FIG. 5B is an ultrasound image of a tendon-only allograft obtained 12 weeks after surgery.

FIG. 5C is an ultrasound image of an IST autograft obtained 12 weeks after surgery.

FIG. 6 is a summary of the measurements of the elongation of rotator cuff tissues during a 50 N applied load.

FIG. 7 is a summary plot of measured tissue stiffness of rotator cuff tissues.

FIG. 8A is a microscope image of the interface between the allograft bone block and the native bone in a bone-tendon allograft 12 weeks after surgery.

FIG. 8B is a microscope image of the interface between the allograft bone block and tendon in a bone-tendon allograft 12 weeks after surgery.

FIG. 8C is a microscope image of the interface between the allograft tendon and the native muscle in a bone-tendon allograft 12 weeks after surgery.

FIG. 9A is a microscope image of the interface between the allograft tendon and the native bone in a tendon-only allograft 12 weeks after surgery.

FIG. 9B is a microscope image of the interface between the allograft tendon and the native muscle in a tendon-only allograft 12 weeks after surgery.

FIG. 10A is a microscope image of the interface between the autograft bone block and the native bone in an autograft 12 weeks after surgery.

FIG. 10B is a microscope image of the interface between the autograft tendon and autograft bone in an autograft 12 weeks after surgery.

### DETAILED DESCRIPTION OF INVENTION

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety.

### 1. Biomaterial

In various embodiments, the invention provides a novel biomaterial for use in rotator cuff repair. The biomaterial includes a bone block attached to a tendon, provided however that the tissues making up the biomaterial are tissues other than knee tissues or ankle tissues. Knee tissues, as defined herein, refer to bone tissues, tendon tissues, and ligament tissues of the knee joint including but not limited to patellar bone, femoral bone, tibial bone, fibular bone, patellar ligament, anterior cruciate ligament, posterior cruciate ligament, medial collateral ligament, semimembranosus tendon, and lateral collateral ligament. Ankle tissues, as defined herein, refer to bone tissues, tendon tissues, and ligament tissues of the ankle joint including but not limited to Achilles tendon, anterior inferior tibiofibular ligament, posterior inferior tibiofibular ligament, anterior talofibular ligament, posterior talofibular ligament, calcaneofibular ligament, calcaneus bone, and talus bone.

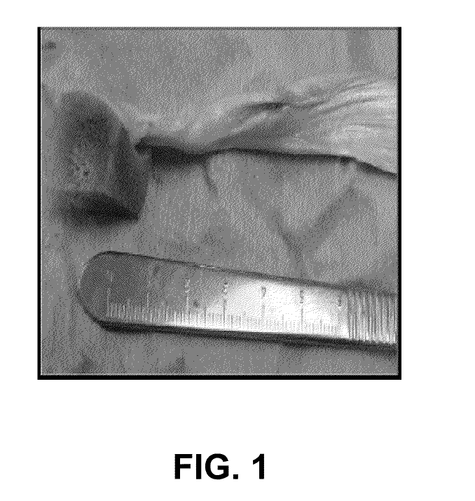

The biomaterial may include bone tissue and tendon tissue from the shoulder girdle and rotator cuff. Non-limiting examples of the biomaterial include a greater tuberosity bone block attached to an infraspinatus tendon, a greater tuberosity bone block attached to a supraspinatus tendon, a proximal humerus bone block attached to a teres minor tendon, and a proximal humerus bone block attached to a subscapularis tendon. A photograph of a non-limiting exemplary biomaterial is shown in FIG. 1, including the bone block and the tendon.

The biomaterial may include tissues harvested from the rotator cuffs and shoulder girdles of human cadavers. During harvest from a cadaver, the tendon, the attachment of the tendon, and the bone block underlying the attachment of the tendon may be harvested as a single intact unit. As a result, the tissue composition and the biomechanical properties of the biomaterial are substantially similar to the composition and biomechanical properties of the injured rotator cuff tissues to be replaced.

The use of the biomaterial in a rotator cuff repair overcomes at least several shortcomings of existing rotator cuff repair techniques. The bone block end of the biomaterial is secured to the native bone of the rotator cuff repair recipient using strong fasteners such as cannulated or interference screws that provide a substantially higher strength of attachment than existing rotator cuff repair techniques. For example, existing rotator cuff repair methods using tendon allografts make use of less secure fixation methods such as pins or sutures through the soft tissue of the tendon allograft to secure the allograft to the native bone of the rotator cuff repair recipient. Because the biomaterial may be derived from cadaverous rotator cuff tissues, rotator cuffs repaired using the biomaterial may function more like a healthy rotator cuff than rotator cuffs repaired using existing allograft materials such as knee-derived tendons or artificial graft materials. Without being bound to any particular theory, because the biomaterial is substantially similar in composition and biomechanical properties to the corresponding properties of the injured rotator cuff tissue prior to injury, any rotator cuff repair that is accomplished using the biomaterial is likely to function essentially the same as a normal uninjured rotator cuff.

The bone block may include any bone tissue making up the shoulder girdle to which any of the tendons of the rotator cuff attach, including but not limited to the proximal humerus as well as the greater tuberosity of the humerus. The shape of the bone block may be determined by any one or more of at least several factors including the shape of the tendon attachment of the biomaterial, the type of fastener to be used to secure the bone block to the native bone tissue, the amount of available bone tissue in the donor cadaver, and the particular surgical instrument used to dissect the bone block away from the donor bone tissue. Non-limiting examples of bone block shapes include a rectangular block, a cylinder, and a pyramid.

The size of the bone block may be determined by any one or more of the at least several factors that determined the shape of the bone blocks described above. If the bone block is a rectangular block, the block may have a square cross-section with dimensions ranging from about 4 mm to about 8 mm, and a length ranging from about 1 cm to about 5 cm. If the bone block is a cylinder, the cylinder may have a diameter ranging from about 4 mm to about 10 mm, and a length ranging from about 1 cm to about 5 cm. If the bone block is a pyramid, the base of the bone block may range from about 4 mm to about 8 mm per side, and the height of the bone block may range from about 1 cm to about 5 cm.

In one embodiment, the dimensions of the bone block and tendon parts of the biomaterial are consistent with the dimensions of the corresponding tendons and insertion footprints of the native rotator cuff structures. As a result, the biomaterial is suitable for use with any existing rotator cuff repair techniques including but not limited to open shoulder surgery, mini open surgery, and arthroscopic surgery and using existing surgical instruments.

### 2. Harvest of Biomaterial

The biomaterial may be harvested from a donor cadaver using methods known in the art. The tendon and bone block to which the tendon is attached may be dissected from the cadaver as a single continuous unit using known methods. The tendon may be separated from the donor muscle to which it is attached as near to the muscle as is practical, in order to dissect the maximum length of tendon for the biomaterial. The bone block may be dissected from the donor bone tissue using known instruments including but not limited to a core cutter, a borer, a bone saw, or a bone drill. In an embodiment, the bone block may be separated from the donor bone using specific standardized instrumentation, resulting in a bone block of a standardized size and shape. The standardized size and shape of the bone block may simplify the procedures and required instrumentation for rotator cuff repairs.

Once the biomaterial has been dissected away from the donor cadaver, the biomaterial may then be processed and preserved for use as allografts using known tissue bank methodologies. The biomaterial may be cell and marrow depleted and washed, and then decontaminated using techniques known in the art including but not limited to washing with solvents including but not limited to ethanol, ethylene oxide, hydrogen peroxide, beta-propriolactone, peracetic acid or glutaraldehyde, or irradiation with gamma radiation, cobalt irradiation, or microwave irradiation.

The decontaminated biomaterial may be used as is within a limited period after the time of harvest, or the biomaterial may be preserved using known techniques including but not limited to freezing at temperatures ranging from about −20° C. to about −80° C. Prior to freezing, the biomaterial may be treated with a cryoprotectant such as dimethyl sulfoxide to minimize damage to any cells within the biomaterial due to freezing. The biomaterial may be thawed prior to use in a rotator cuff repair.

### 3. Methods of Using Biomaterial

The biomaterial may be used in the repair of a rotator cuff using existing surgical methods including but not limited to open shoulder surgery, mini open surgery, and arthroscopic surgery. In an exemplary embodiment of a rotator cuff repair, the injured tissue of the rotator cuff may be debrided to remove damaged or necrotic tissue. After debridement, the lateral section of the injured or ruptured tendon may be removed by removing a section of the native humerus surrounding the attachment of the tendon, forming a recipient bed in the native bone into which the bone block of the biomaterial may be inserted. The native bone tissue may be removed using surgical instruments known in the art including but not limited to a core cutter, a borer, a bone saw, or a bone drill. In an exemplary embodiment, the native bone tissue is removed using the same instrumentation used to dissect the biomaterial from the donor bone tissue. In this exemplary embodiment, because the same instrumentation is used for both the dissection of the biomaterial bone block and the removal of the native bone tissue, the process of fitting the bone block to the recipient bed is simplified.

Once the injured tendon has been removed and the recipient bed has been formed, the bone block of the biomaterial may be inserted into the recipient bed and fixed into place using any suitable fixation device known in the art. Non-limiting examples of suitable fixation devices include cannulated screws, interference screws, anchors, pins, or suture bridges.

The tendon end of the biomaterial may be cut to an appropriate size depending on any one of several factors including but not limited to the individual morphology of the recipient patient, the extent of the rotator cuff injury, and the desired graft splice type. Non-limiting examples of suitable splices of the tendon end of the biomaterial include tendon-tendon grafts and tendon-muscle grafts. The tendon end of the biomaterial may be joined to the native tendon or native muscle tissue using any method known in the art including absorbable sutures, non-absorbable sutures, and surgical staples. Sutures may be used to join the tendon end of the biomaterial to the native muscle or native tendon in any suitable suture pattern known in the art. Non-limiting examples of suitable suture patterns include a vertical mattress pattern, a horizontal mattress pattern, a crossed mattress pattern, a single running pattern, an interrupted running pattern, a running locked suture pattern, or a pulley suture pattern. The particular type of splice and method of joining may be selected based on at least one of several factors including but not limited to the location of the rotator cuff injury, the extent of the injury, the desired strength or stiffness of the join, and the desired pattern or rate of healing of the graft using the rotator cuff repair. In an exemplary embodiment, the tendon end of the biomaterial is joined to the native tendon using a tendon-tendon graft joined by non-dissolvable sutures in a vertical mattress suture pattern.

Once the rotator cuff repair is completed, the bone block of the biomaterial may be incorporated into the native bone tissue, and the tendon end of the biomaterial may be integrated with the native tendon or muscle tissue, forming organized repair tissue and ultimately an attachment that possesses substantially similar tissue morphology and biomechanical properties as uninjured native rotator cuff structures.

### Method of Selecting Biomaterial for Rotator Cuff Repair Procedures

An embodiment of the invention provides a method of selecting a biomaterial for a rotator cuff repair. In this embodiment, the biomaterial is selected in order to match the particular tissues making up the biomaterial to the injured native tissue in the recipient rotator cuff to be repaired. The biomaterial may be selected that includes a rotator cuff tendon. If the type of tendon that is injured in the rotator is identified, the biomaterial may be selected so that the type of tendon included in the biomaterial matches the injured tendon type.

For example, if the recipient rotator cuff is found to have a ruptured infraspinatus tendon, a biomaterial that includes a greater tuberosity bone block attached to a infraspinatus tendon may be selected for the repair procedure. By selecting a biomaterial that includes tissue types that are substantially similar to the injured tissues in the recipient rotator cuff, the biomaterial may function in an essentially similar manner to the tissue replaced by the biomaterial. The various tendons of the rotator cuff may possess variations in the abundance, structure, distribution, and orientation of the collagen fibers, the composition of the tendon's matrix, and the length and thickness of the tendon that may individually or in combination influence the function of a the tendon in the rotator cuff.

Without being bound to any particular theory, the more similar the biomaterial is to the type of tendon to be repaired in the rotator cuff, the more likely that the biomaterial will function in a similar manner to the tendon prior to injury. In an embodiment, the biomaterial selected for a rotator cuff repair may be any rotator cuff tissue selected from a greater tuberosity bone block attached to a infraspinatus tendon, a greater tuberosity bone block attached to a supraspinatus tendon, a proximal humerus bone block attached to a teres minor tendon, and a proximal humerus bone block attached to a subscapularis tendon. In an exemplary embodiment, the biomaterial that includes the particular tendon to be repaired in the rotator cuff is selected for the rotator cuff repair procedure.

### Example 1

### Efficacy of Alloqraft Repair of Rotator Cuff in a Canine Shoulder Model

To assess the efficacy of using an embodiment of the biomaterial in a bone-tendon allograft technique for rotator cuff repair, the following experiments were conducted. Four adult purpose-bred mongrel dogs underwent surgical rotator cuff repair using the bone-tendon allograft technique and other techniques as described below. In particular, the bone-tendon allograft technique was compared to an existing tendon-only allograft technique to evaluate the clinical efficacy for the bone-tendon allograft technique for repair of the rotator cuff in a canine model. All dogs underwent bilateral infraspinatus tendon (IST) partial tenectomies, except as noted below. The defect introduced by the partial tenectomy was then repaired by either the bone-tendon (B-T) allograft technique (n=3), or by a tendon-only allograft (n=3). All procedures were approved by the institutional ACUC.

All allografts were obtained from canine cadavers and processed by human tissue banks using standard processing methods. The allografts included infraspinatus tendon, supraspinatus tendon with bone block, and teres minor tendon with bone block.

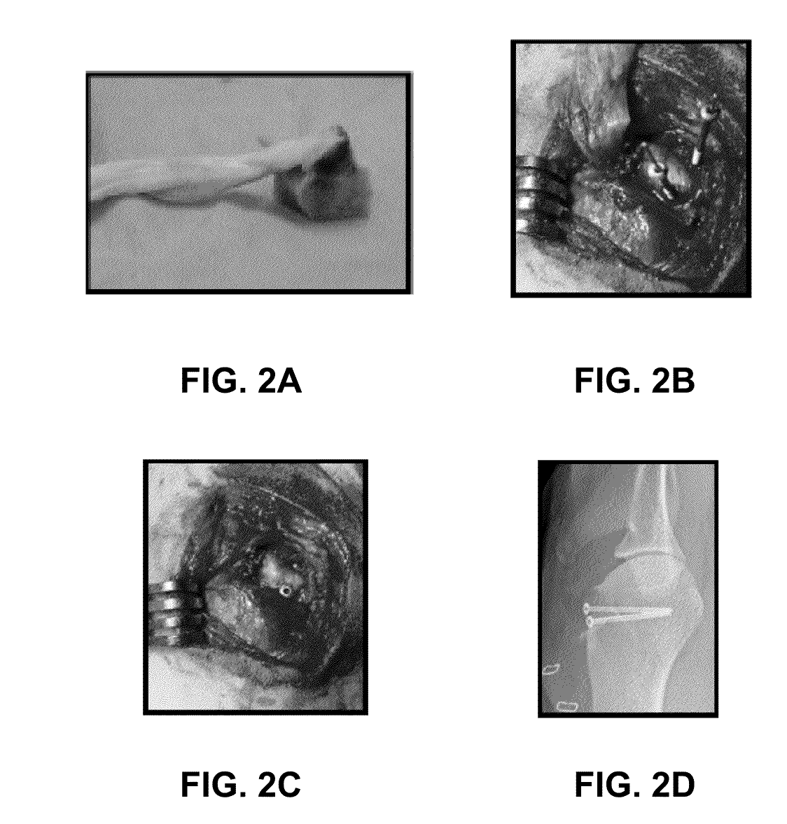

A photograph of a representative B-T allograft used in the B-T allograft technique is shown in FIG. 2A. In the B-T allograft technique, the native IST was attached to the tendon end of the B-T allograft using a suture bridge as shown in FIG. 2B. The bone block end of the B-T allograft was affixed to the native bone using cannulated screws, as shown in FIG. 2C. FIG. 2D is a representative post-operative radiograph image of the shoulder joint following the B-T allograft repair obtained twelve weeks after the surgery.

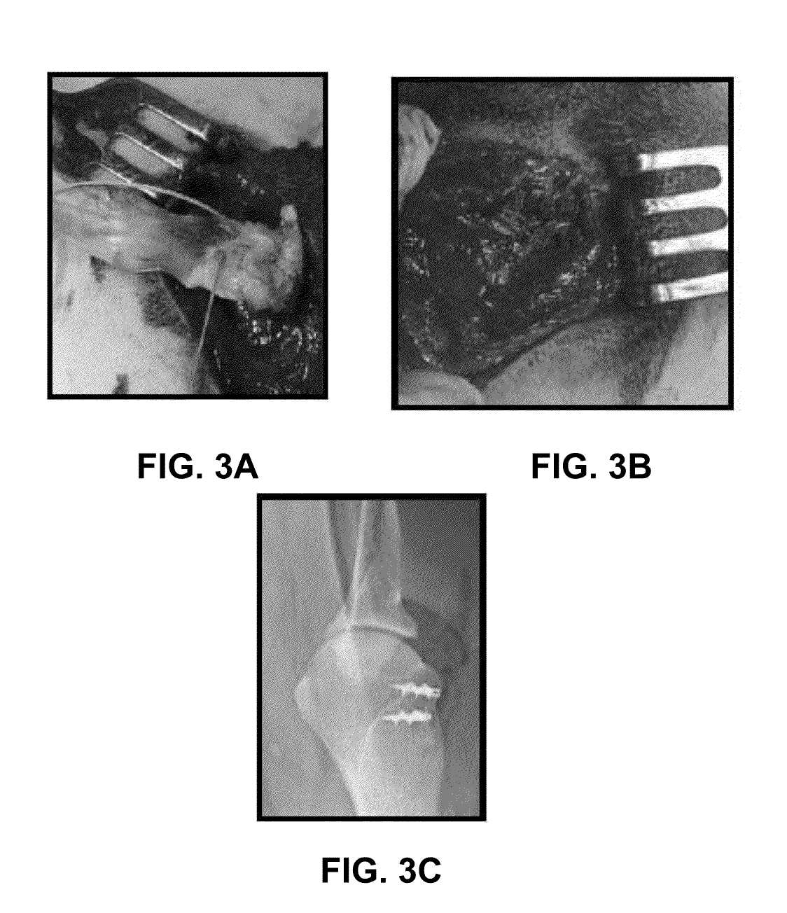

In the tendon-only allograft, both ends of the tendon allograft were spliced into the tenectomized IST using suture bridges. The tendon allograft was inserted as shown in FIG. 3A, and sutured to the native IST as shown in FIG. 3B. FIG. 3C is a representative post-operative radiograph image following the tendon-only allograft implantation obtained twelve week after the surgery.

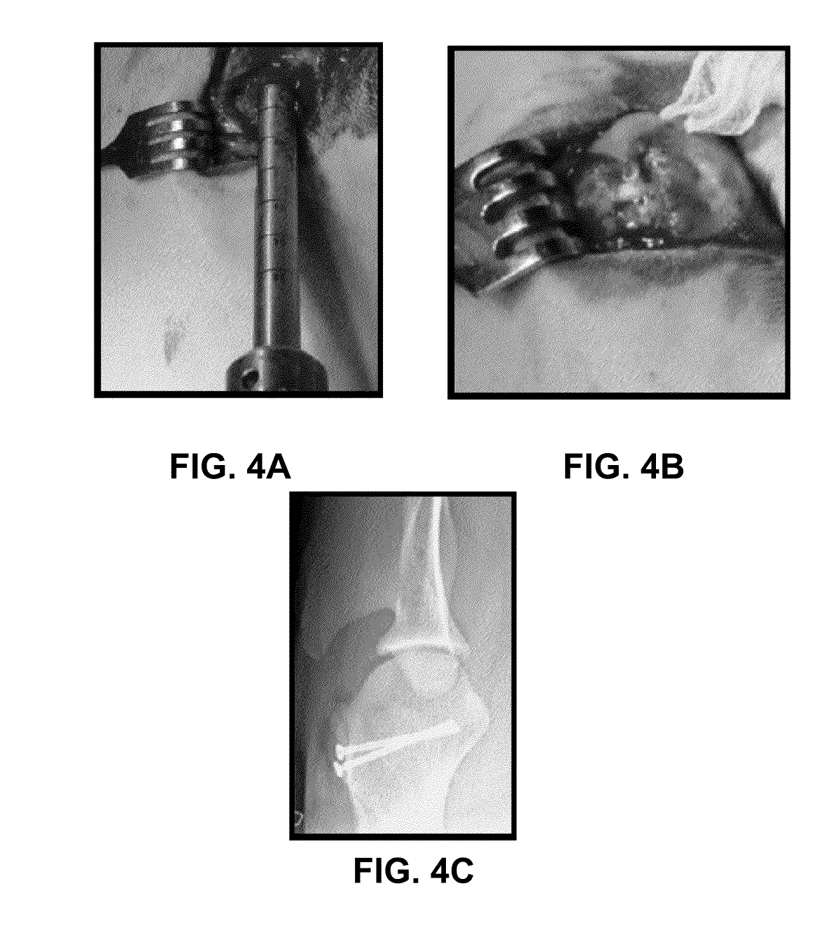

As a positive control, two rotator cuffs were subjected to in situ autografts of the IST. In the autograft procedure, the native IST with a bone block at its insertion was elevated as shown in FIG. 4A, and then replaced using cannulated screw fixation as shown in FIG. 4B. A representative postoperative radiograph image obtained 12 weeks after the autograft procedure is shown in FIG. 4C. The shoulders undergoing the autograft procedure did not undergo the initial IST tenectomy, so no attachment of the autograft on the tendinous end was necessary.

Following the surgical procedures described above, all dogs were housed in individual cages and allowed unrestricted mobilization for 12 weeks. After 12 weeks, functional assessments of the repaired rotator cuffs were performed on all dogs. In addition, radiograph and ultrasound images of both shoulders of all dogs were obtained. Upon completion of the functional assessments and imaging, all dogs were euthanized, and non-destructive biomechanical and histological measurements were obtained for all shoulders.

For functional assessment, a previously validated lameness assessment system was used to score each forelimb of each dog at 12 weeks post-surgery on a scale ranging from zero to five. A score of zero indicated normal functional use and a score of five indicated no functional use of the limb. All forelimbs of all dogs recovered fully post-operatively and had normal limb function for all surgical treatments performed (results not shown).

Cranial-caudal and medial-lateral radiographs of all shoulders were obtained at twelve weeks post-surgery to assess the integrity of the implants and the graft unions, as well as to assess any radiographic pathologies. All radiographic images, as shown in FIG. 2D (B-T allograft), FIG. 3C (tendon-only allogaft), and FIG. 4C (autograft) exhibit evidence of good bone healing and integration of the bone blocks in both the B-T allograft and autograft groups. No evidence of implant failure, migration, infection or shoulder arthritis was observed in any shoulder based on the analysis of the radiographic images.

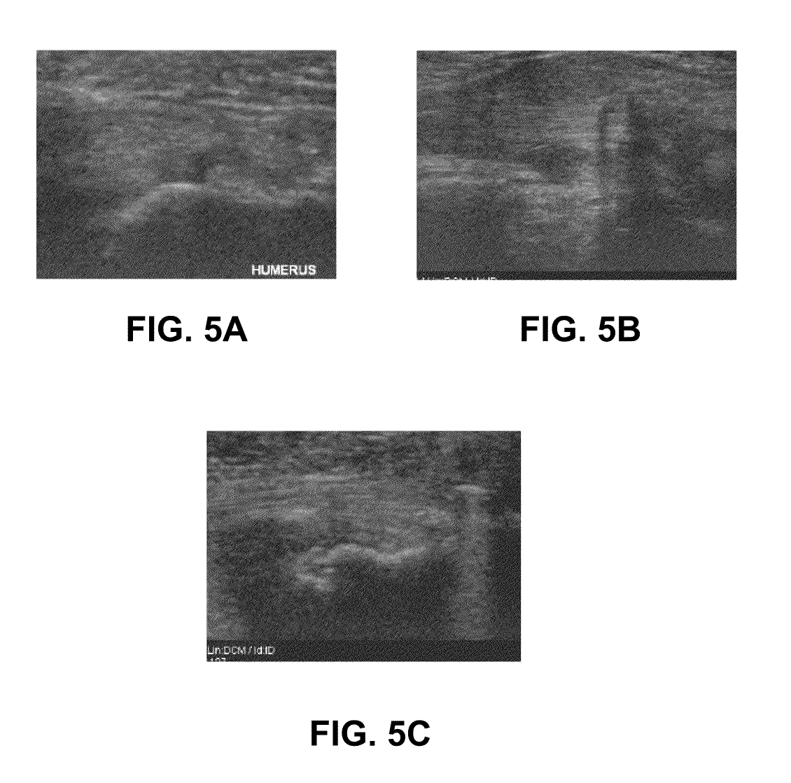

Ultrasound images of all shoulders of all dogs were also obtained at twelve weeks post-surgery to assess IST architecture and integrity from the tendon's origin to its insertion. FIG. 5A is a representative ultrasound image for a B-T allograft, showing no abnormalities in the bone-tendon attachment. An ultrasound image of the tendon-only allograft is shown in FIG. 5B; the ultrasound images indicated that the attachment of tendon to bone included tissue with a disorganized and heterogenous echogenic appearance. No abnormalities in the bone-tendon attachment were observed in the ultrasound images of the shoulders within the autograft group; a representative ultrasound image is shown in FIG. 5C.

Biomechanical testing of the rotator cuff tissues were performed after the euthanasia of the dogs. The IST bone-tendon-muscle complex was excised en bloc, placed in a specialized jig and tested by a load applied along the longitudinal axis of the complex. The load was applied to the complex with gradually increasing force in order to stretch the complex at a constant rate of 0.10 mm/sec. All complexes were tested up to a lateral pull of 50N (walking load for a dog) or up to a resulting elongation of 2 mm at the bone-tendon or the tendon-muscle junction, whichever occurred first. Optical markers were placed between the bone-tendon repair site, on the IST, and on the tendon-muscle junction. The degree of elongation of the bone-tendon-muscle complex was measured as the change in distance between the markers as determined by an optical tracking system (NDI Optotrak, ON, Canada). The optical tracking data were synchronized with the load data. As a negative control, the biomechanical measurements described above were performed using normal bone-tendon-muscle complexes excised from age, weight, and breed matched normal canine cadaveric shoulders (n=4).

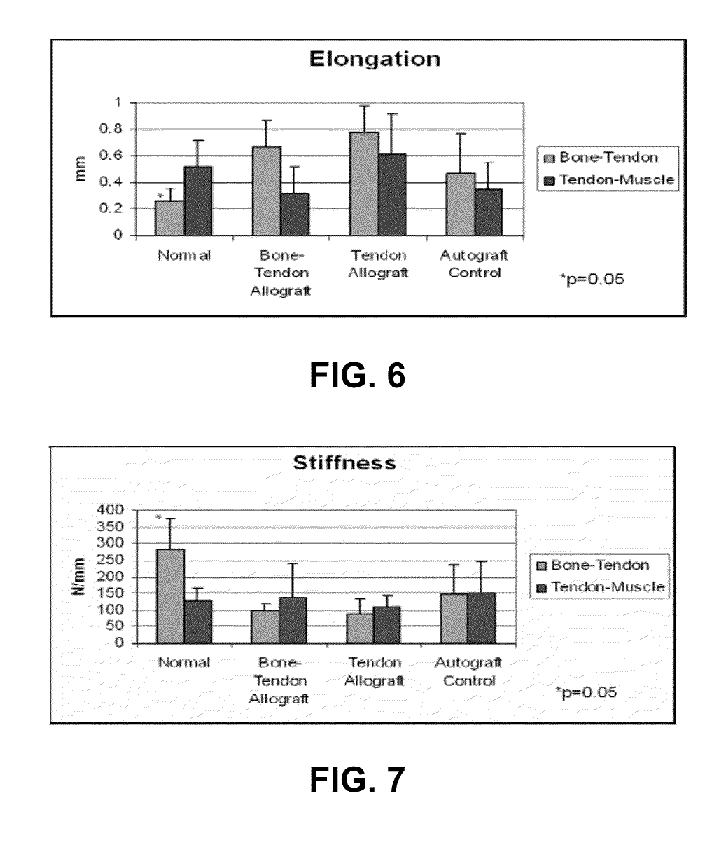

FIG. 6 summarizes the measured tissue elongation at 50N of applied load within the bone-tendon junction and the tendon-muscle junction for all groups tested. None of the treatment groups reached the clinically relevant critical elongation of >2 mm at 50 N of applied load.

The stiffness of all IST bone-tendon-muscle complexes was assessed by calculating the rate of elongation of each junction of the complexes with respect to the applied load between 20N and 30N. FIG. 7 summarizes the stiffness of the junctions of all groups tested. The stiffness of the bone-tendon junction was significantly higher (p<0.05) in the normal group than in all other groups. Measured tendon-muscle stiffness was not significantly different among any of the groups tested. The stiffness of both the bone-tendon junction and of the tendon-muscle junction were slightly higher for the B-T allograft group compared to the tendon-only allograft group, although these differences were not significantly different.

Histologic sections of each IST complex were obtained for all shoulders of all groups. Each section was decalcified and stained using haematoxylin and eosin. The stained sections were assessed for cell and tissue morphology by a pathologist blinded to treatment.

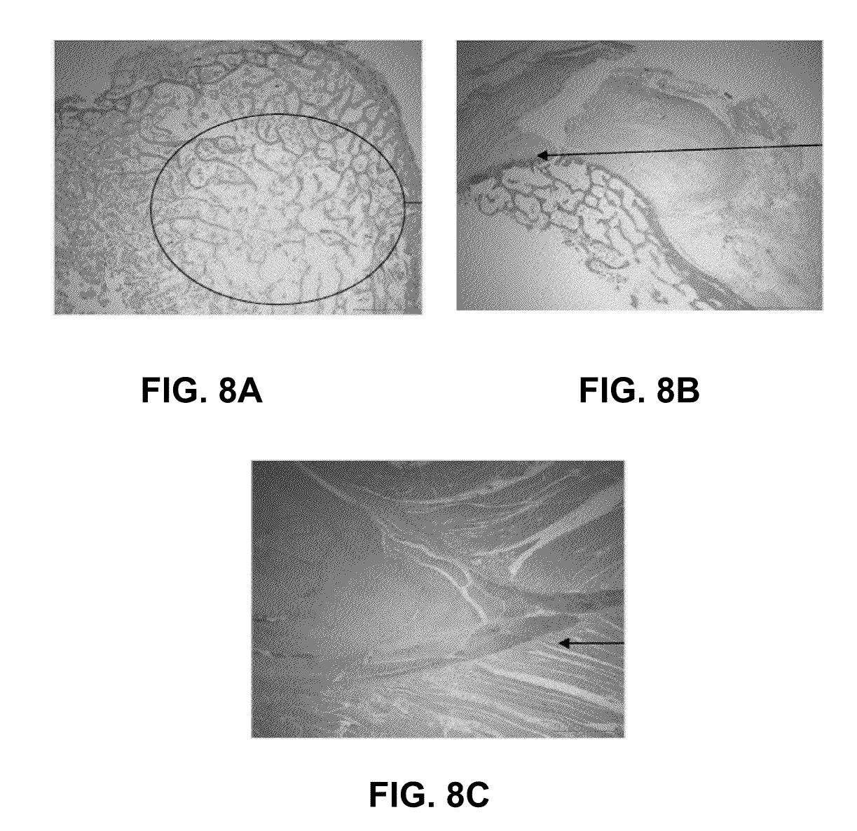

FIGS. 8A-8C are representative microscopic images of the B-T allograft histologic sections. The pathologist's assessment of the interface between the allograft bone block and the native bone tissue, as shown in FIG. 8A, indicated good incorporation of the bone block into the native bone with no indication of an inflammatory response and minimal gap formation. The bone-tendon junction, shown in FIG. 8B, maintained a normal tendon attachment. The muscle-tendon junction, as shown in FIG. 8C, maintained normal tissue integrity and good integration of the allograft tissue with the native tissues.

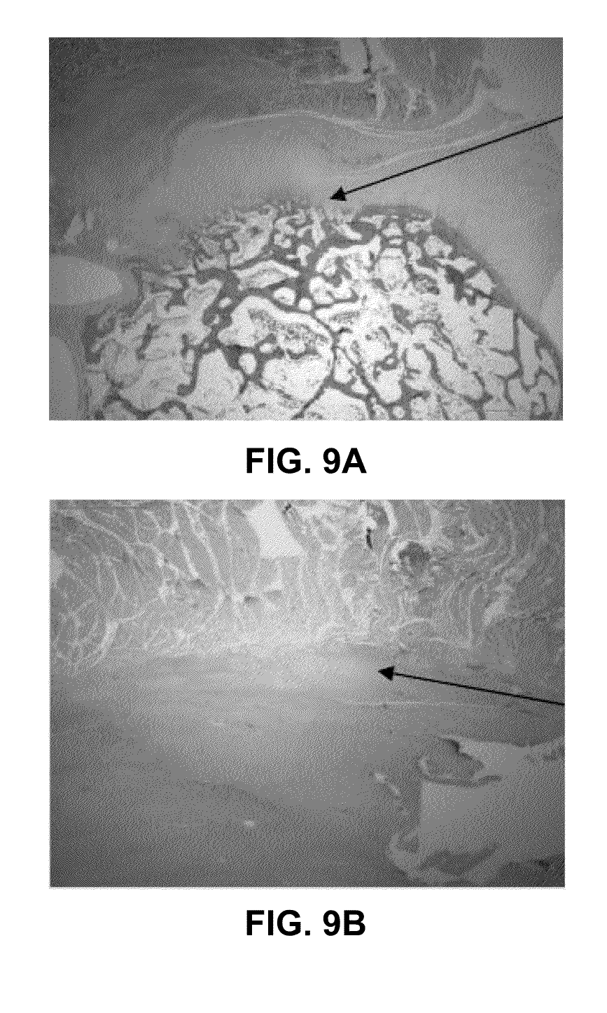

FIG. 9A and FIG. 9B are representative microscopic images of the tendon-only allograft histologic sections. The attachment of the allograft tendon to the host bone at the insertion site ranged from loose connective tissue to robust fibrous tissue, as shown in FIG. 9A. Areas of graft degeneration and chondroid metaplasia were noted in some sections from this group. The muscle-tendon junction, shown in FIG. 9B, maintained normal tissue integrity and showed good integration of the allograft tendon tissue with the native muscle tissue.

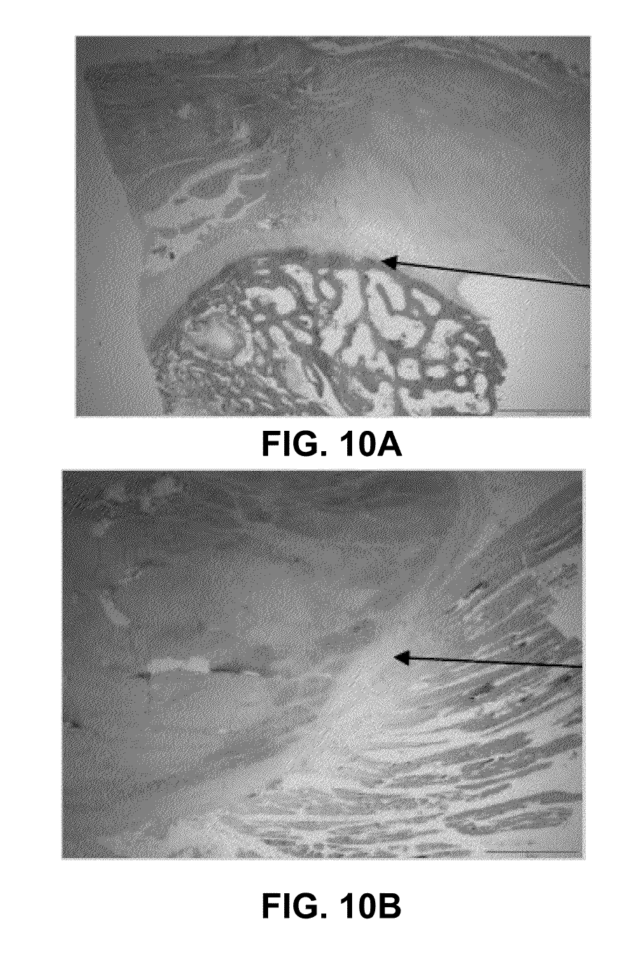

Representative microscopic images of the autograft histologic sections are shown in FIG. 10A and FIG. 10B. The autograft histologic sections were very similar in appearance to the B-T allograft sections. The integration of the autograft bone block into the native bone showed no indication of gap formation or inflammatory response, as shown in FIG. 10A. The bone-tendon junction maintained a normal tendon attachment, as shown in FIG. 10B.

The results of these experiments indicated that the B-T allograft technique may be a viable surgical option for rotator cuff repair. The functional abilities of the shoulders and limbs of all subjects undergoing B-T allograft surgeries were not compromised. Based on radiographs and histologic findings, bone integration and healing of all subjects undergoing B-T allograft surgery were excellent with no evidence of immunological reaction or gap formation. Further, the bone-tendon attachment maintained normal tissue architecture and integrity with evidence of cellular repopulation for the B-T allograft repairs. By contrast, subjects undergoing tendon-only allograft repairs showed attachment of the allograft tendon to the native bone via disorganized fibrous repair tissue only. Based on biomechanical measurements, the B-T allografts were superior to the tendon-only allografts in both degree of elongation and stiffness under loading.

While the invention has been explained in relation to exemplary embodiments, it is to be understood that various modifications thereof will become apparent to those skilled in the art upon reading the description. Therefore, it is to be understood that the invention disclosed herein is intended to cover such modifications as fall within the scope of the appended claims.

What is claimed is:

1. A biomaterial comprising an intact bone-tendon unit for the repair of a rotator cuff, wherein the bone-tendon unit comprises a bone block attached to a tendon, provided however that the intact bone-tendon unit consists of tissues other than knee tissues or ankle tissues.

2. The biomaterial of claim 1, wherein the tendon is a rotator cuff tendon selected from an infraspinatus tendon, a supraspinatus tendon, a teres minor tendon, or a subscapularis tendon.

3. The biomaterial of claim 1, wherein the bone-tendon unit is selected from a greater tuberosity bone block with an infraspinatus tendon, a greater tuberosity bone block with a supraspinatus tendon, a proximal humerus bone block with a teres minor tendon, or a proximal humerus bone block with a subscapularis tendon.

4. The biomaterial of claim 1, wherein the rotator cuff comprises at least one injured tendon, and wherein the tendon of the biomaterial is substantially the same type of tendon as the at least one injured tendon.

5. The biomaterial of claim 1, wherein the bone-tendon unit is harvested from a human cadaver, depleted of cells and marrow, and decontaminated.

6. The biomaterial of claim 1, wherein the bone block has a shape selected from a rectangle shape, a cylinder shape, or a pyramid shape.

7. The biomaterial of claim 6, wherein the bone block has a rectangle shape, wherein the rectangle shape has cross-sectional dimensions ranging from about 4 mm to about 8 mm, and a length ranging from about 1 cm to about 5 cm.

8. The biomaterial of claim 6, wherein the bone block has a cylinder shape, wherein the cylinder shape has a diameter ranging from about 4 mm to about 8 mm, and a length ranging from about 1 cm to about 5 cm.

9. The biomaterial of claim 6, wherein the bone block has a pyramid shape comprising a base, wherein the base has sides ranging in size from about 4 mm to about 8 mm, and a height ranging from about 1 cm to about 5 cm.

10. The biomaterial of claim 1, wherein the biomaterial is compatible with a surgical technique selected from open shoulder surgery, mini open surgery, and arthroscopic surgery.

11. A method of selecting a biomaterial for use in a repair of a rotator cuff, wherein the biomaterial comprises an intact bone-tendon unit, wherein the bone-tendon unit comprises a bone block attached to a tendon, the method comprising selecting a biomaterial in which the tendon is a rotator cuff tendon.

12. The method of claim 11, wherein the rotator cuff tendon is selected from an infraspinatus tendon, a supraspinatus tendon, a teres minor tendon, or a subscapularis tendon.

13. The method of claim 11, wherein the bone-tendon unit is selected from a greater tuberosity bone block with an infraspinatus tendon, a greater tuberosity bone block with a supraspinatus tendon, a proximal humerus bone block with a teres minor tendon, or a proximal humerus bone block with a subscapularis tendon.

14. The method of claim 11, wherein the method further comprises identifying at least one injured tendon in the rotator cuff to be repaired before selecting the biomaterial.

15. The method of claim 14, wherein the rotator cuff tendon of the biomaterial is essentially the same type of tendon as the injured tendon.
